(19) United States
(12) Patent Application Publication
(10) Pub. No.: US 2023/0056628 A1
(43) Pub. Date: Feb. 23, 2023

(54) HEALING TAPE

(71) Applicant: ARTHUR H. KRUGLER, Grass Valley, CA (US)

(72) Inventor: ARTHUR H. KRUGLER, Grass Valley, CA (US)

(21) Appl. No.: 17/979,307

(22) Filed: Nov. 2, 2022

Related U.S. Application Data
(63) Continuation-in-part of application No. 16/873,656, filed on Jun. 1, 2020, now abandoned.

(51) Int. Cl. A61F 13/02; A61F 13/00

(52) U.S. Cl. CPC A61F 13/0266; A61F 13/0253; A61F 13/00063

(57) ABSTRACT

Disclosed is an adhesive wound healing tape comprised of a water vapor permeable backing and adhesive that can be applied over a wound and remain in place throughout the healing process. Preferably, the tape is applied over a wound without gauze bandage or non-stick gauze material and compresses opposing sides of the cut into one another to promote faster healing with a less visible and often invisible scar.

### CROSS-REFERENCE TO RELATED APPLICATIONS

This application is a continuation-in-part of U.S. patent application Ser. No. 16/873,656, filed on Jun. 1, 2020, incorporated herein by reference in its entirety.

### FIELD OF THE INVENTION

The following invention relates to wound dressings to hold skin together during healing. More particularly, this invention relates to wound dressings in the form of flexible tape having an adhesive on one side and a porous character.

### BACKGROUND OF THE INVENTION

The idea of using bandages to protect a wound has been known for hundreds of years. More recently the use of an adhesive tape that includes a non-stick pad to allow easy removal and replacement has been popular. It is sold for commercial purchase at pharmacies and drug stores under the brand name “BAND-AID,” a registered trademark of Johnson & Johnson located in New Brunswick, N.J., or other names. Hospitals use a gauze (cotton) dressing held in place with “Surgical Tape” to cover the wound and absorb blood and wound emissions when a cut or wound is bleeding. The blood can seep into and be captured by the gauze.

During healing of an open wound or cut, the body forms a solid red “scab” of clotted blood and lymph. Subsequent to scab formation, the body is able to heal the wound beneath the scab where the scab is in essence the body's natural healing barrier to block ingress of pathogens. Unfortunately for wounds and cuts, the scab heals leaving a near permanent white scar and the larger, deeper, or longer the cut the larger will typically be the scab and as well, the scar. It would be an advantage to devise a superior method to 1) protect a wound during healing, 2) that would reduce the size of, or eliminate the resulting scar, and 3) it would also be an advantage to devise a superior method to reduce pain during healing and also 4) that would reduce the healing time.

One reason for this is that common adhesive “BAND-AIDS” and bandages with a gauze for fluid absorption successfully provide a covering over the wound, but they do not block bodily fluid flow out through the surface of the wounded skin. Further, for cuts, they do not compress opposing sides of the cut into one another. If flesh is cut, even deeply, and then it is compressed into itself, the flesh and skin surface will re-bond within minutes of the skin being cut. However, the tensile strength of the growing bond is initially almost zero. It takes several days for the bond strength to grow so that the wound is as strong as the skin was prior to the cut being made into the skin.

For cuts, especially for deep and or long cuts, another common practice is to suture, or stitch, the wound together. Such wounds can be caused by accident or injury, or be part of a surgical procedure, and present the same wound closing need. Sutures are better than an adhesive bandage because they compress the skin on one side of a cut line into the skin on the other side of a cut line. However, the suture closure force is uneven, tending to be very high (even too high) at the suture point and significantly lower (even too low) at midpoints between the suture points. The suture forces the cut skin to close where the suture pressure is applied. Compared to just putting a gauze bandage over the cut line, a suture will achieve a superior closure including a much smaller scar. A more even closure force than that provided by sutures to close wound margins together can best support blood vessel regrowth spanning the wound, and avoid excessive compression of the body tissues which can inhibit healing blood flow. While more “stitches” closer together can be one option, this is time consuming and difficult to precisely put in place.

Sutures do have an advantage over leaving the wound open and uncovered, and even over covering the untreated, unstitched wound with cloth, gauze or “BAND-AID” because the sutures do compress two sides of the wound directly underneath the suture. Unfortunately, sutures only compress skin directly beneath each suture but no elsewhere between sutures along the length of the wood. This fact causes a very undesirable condition which increases healing time from the ideal and possible cure time of 4-7 days to 4-6 weeks and even longer. First, where the skin is compressed within the suture, capillaries are also compressed which reduces or restricts blood flow which is necessary for cells to flourish and heal. Second, between the sutures, a scab of clotted blot is formed which also prevents blood circulation and delays healing. Third, the scab of clotted blood is replaced by our bodies with white scar tissue which also prevents blood circulation for years. Fourth, a location is needed above the skin for clots to form and to allow pink skin cells to fill any gap between the two sides of the cut.

Unfortunately, the scar formed, especially for large cuts such as are common during surgery, include a scar along the cut line and also scar dots where the suture and needle penetrated the skin to hold the opposite sides of the cut skin together. It would be advantageous if a new method for securely holding the skin on opposite sides of a wound could be devised to hold the skin on opposing sides of a cut into one another and to immobilize the opposing sides of a cut line in a proximal condition throughout the healing process.

Another benefit of sutures over “BAND-AIDS” is that they significantly close the wound for cuts, whether from surgery or from an accident. Closing the opposing sides of a wound reduces the area of internal body exposed to infectious microbes. But even sutures only compress the skin into itself at the locations of the stitches. Between the stitches, the flesh and skin pucker open and where each suture penetrates the skin twice to pull the cut together, the suture penetration provides another path into the interior of the body that microbes can follow.

It would be advantageous if there could be created a new technology that could entirely block the passage of microbes into the wound in part due to blocking access to the wound site, but also because the new technology could compress opposite sides of a cut line into one another without penetrating sutures. In this manner, one can imagine that a new technology might be able to do a superior job of compressing opposite sides of a cut into one another and at the same time, do so without requiring the addition of sutures and the resulting skin penetration holes where the suture penetrates into the body.

In another application, elderly people have thin and fragile skin. Often, sutures will cause the thin skin to tear, preventing their use. Applying “BAND-AIDS” do not promote rapid healing or avoid scars. It would be an advantage if a new method were devised that could more gently hold onto the fragile skin of elderly people and yet compress the opposing sides of a cut into one another.

At the other end of the spectrum, children often have a significant aversion and/or fear of needles, including suture needles. Thus, alternatives to suturing needles allow for wound closure with less trauma and less need to manage patient comfort and fear, especially for young children. Also, wound closure alternatives for young children have the opportunity to reduce or eliminate scarring, and its attendant disadvantages.

In another application, people have sores that cover a small area of skin. These can come from for example, diabetes, or they could result from a fall to the ground where a knee becomes scraped and the skin over a larger area than a typical cut is grated and raw. Such a wound requires a protective barrier to block microbes from entering the wounded skin and to protect the sensitive area.

Washing and bathing can re-open sores and wounds and reverse the healing that was previously achieved. This can in some circumstances result in a situation where a sore on the skin persists virtually indefinitely. Sutures cannot close such a sore and typical bandages and band aids do not provide rapid healing.

A common problem with original adhesive bandages is that the tape material is not water vapor permeable. This results in the skin becoming overly hydrated, white, puffy and would not heal. When one has been in water for a long period of time, the skin becomes like dried “prunes,” all moist and wrinkly. Many adhesive bandages get around this problem by punching a number of holes through the water vapor impermeable tape. The water vapor coming from the body/skin can pass out through the holes in the tape and if there are sufficient holes, then the skin will not accumulate the water vapor and won't become overly hydrated and “prune-like.” Applying this type of tape with holes directly over the cut or wound provides a path for blood to escape and microbes to enter. Rather than holes in the tape it would be advantageous if a tape that simply allowed water vapor and blood/bodily fluids to pass through were used.

With various bandages, a healing agent can often be applied to the wound and then the gauze covering can be applied over the wound with the healing agent and a surgical tape can hold the gauze with healing agent over the wound during healing. Various kinds of bandages are used and “BAND-AID” is but one brand of numerous styles of adhesive bandages.

Most often, the healing agent is applied to the wound separately of the bandage. For a few bandage types, various healing agents including honey, and other typically anti-microbial agents can be applied to the gauze that is then placed over the wound with the surgical tape holding the gauze impregnated with the healing agent, in place over the wound during the healing process. Unfortunately, this method fails to hold opposing sides of a cut together allowing for a larger scar to result.

Anti-bacterial agents can be applied to the surface of “healing tape” as a thin stripe which will be over the cut or skin problem while healing. In the case of open sores, it would be advantageous if a new healing tape could be created that would at once, provide a barrier to infectious microbes, a pathway for water vapor, and a healing agent that could persistently treat a chronic sore. In essence, it would be advantageous if a new sort of tape could be developed where the tape is in essence, a temporary replacement for skin that can remain in place for a week or two while a wound heals.

For this to work the tape would need to be somewhat liquid water resistant. In other words, if a cut is on the tip of a finger, then when the hands are washed whatever bandage is on the tip of the finger is going to get wet. Typical “BAND-AID” style adhesive bandages wind up with the gauze becoming soaked with water and requiring replacement. Having a skin-like tape that is sufficiently flexible and yet water resistant and water vapor permeable and able to remain affixed to the skin to protect a wound for a week during healing is an object of the present invention. This water resistant aspect is important for the device whether the wound happens to be a cut, tear, scrape, sore or other problem.

### SUMMARY OF THE INVENTION

With this invention, a wound dressing is provided in the form of a thin flexible layer of material with an adhesive on one side thereof. The thin flexible layer of material forms a tape which can span a wound and contact skin on either side of the wound to hold margins of the wound together to facilitate healing. The tape is also porous to allow for the wound to dry naturally and to allow any blood seeping from the wound to pass into the porous material forming the tape and clot therein.

While the adhesive could be any of a variety of different adhesives, one embodiment contemplates using a non-polymerizing adhesive which acts on contact, and does not require a chemical reaction to occur for bonding properties to be present. One example of such a non-polymerizing adhesive which could be used in one embodiment is a methacrylate adhesive. The adhesive is typically provided over an entire lower surface of the tape. In this fashion, the lower surface of the tape can contact skin and hold skin together on opposite sides of a wound, on all available surface area of the lower surface of the tape. The adhesive, as well as the tape itself, is preferably biocompatible and also preferably hypoallergenic in various embodiments, so that the adhesive avoids biological interactions with an individual using the healing tape of this invention.

The porosity of the material providing the thin layer which makes up the tape is preferably selected to be a greater porosity than a porosity of human skin. In this way, the tape does not cause moisture to be retained in the skin to a greater extent than the human skin itself would hold such moisture. Rather, with a greater porosity than human skin, moisture on or in the human skin will migrate through the material from the lower surface toward the upper surface (when the skin is exposed to unsaturated air at least), following a basic principle that moisture tends to migrate from locations of greater liquid saturation to lesser liquid saturation (e.g. from wet to dry).

Such porosity has a variety of benefits. First, blood seeping from the wound (and other bodily fluids) will not be contained below the tape. Rather, such bodily fluids will migrate into the porosity of the tape. Preferably, the tape is sufficiently closing the wound that such liquid flows are relatively small, and such bodily fluids can reside within the porosity of the tape itself, between the lower surface and the upper surface. If any bodily fluids migrate entirely through the porous thin layer of material to the upper surface, such liquids could dry on this surface, or could be wiped off periodically. Second, when washing, bathing, swimming and other water contact is encountered, the water can pass through the material for cleaning. Then, when contact with the water ceases, the residual water on the skin and within the material can migrate to the upper surface and evaporate. The skin is thus kept in a desirable mostly dry state that is not inhibited detrimentally by the healing tape.

In the case of blood flows from the wound under the tape, the blood will tend to coagulate into a scab, as the tape is closing the wound sufficiently that only a very low flow of blood occurs, and such a low flow rate, as well as the dryer environment outside of the skin, promotes coagulation and scab formation. This scab formation is preferably primarily within the porous tape itself between the lower surface and the upper surface. The body fills in any opening between sides of the cut with new pink skin cells, rather than white scar tissue cells. As one option, a coagulating agent can be provided upon or within the material forming the tape, which can promote hardening of blood into a scab within the porous material forming the healing tape. As one option, the adhesive has both bonding properties and coagulation properties.

The tape can be provided on a continuous roll having a constant width, and then be cut off at desired lengths, or can be perforated to facilitate tearing at standardized lengths. The tape can be placed with a length of each strip of tape perpendicular to a length of the wound, or parallel to a length of the wound, depending on whether the wound needs greater closing force applied by the tape to close the wound and keep it closed, or if a wound is generally holding closed already and primarily needs protection from further damage and absorption of blood and other bodily fluids discharged from the wound. For some wounds, more than one strip of tape can be utilized either perpendicular to the wound length or parallel with the wound length, or both.

The tape can be used in conjunction with sutures, or as a replacement to sutures, or on wounds that do not require sutures, as a wound dressing. Beneficially, the tape adheres to skin on either side of the wound over a relatively large surface area, so that unnatural tugging on the skin by the tape is avoided or greatly minimized. Skin of the elderly, especially on hands and arms, can become quite thin. Such skin is difficult or impossible to suture. Furthermore, many elderly individuals are taking blood thinners which can exacerbate bleeding from wounds. Utilizing the tape of this invention, the thin skin of the elderly can be effectively held to close wounds and allow blood and bodily fluids to be discharged from the wound and contained within the porous material forming the tape.

Other details of this invention are illustrated with reference to various examples including a healing tape, comprising in combination a thin flexible layer of material extending between lateral edges spaced apart by a width of the tape, and extending between opposite ends spaced apart by a length of the tape, and with the material having a lower surface parallel with and spaced from an upper surface by a thickness of the tape, wherein a non-polymerizing adhesive is located upon the lower surface, the adhesive being biocompatible with placement adjacent to human skin, the adhesive able to hold to human skin strong enough to avoid un-bonding from human skin when forces associated with wound margin spreading are encountered, and the material having a porosity greater than an average porosity of human skin, such that fluids such as blood are drawn out of the skin and into the material forming the tape.

Options for an invention such as that disclosed above include that adhesive is hypoallergenic, that the adhesive is a non-polymerizing adhesive, that the adhesive is a contact adhesive which releasably bonds to human skin, that the adhesive includes a methacrylate therein, that the material forming the tape has a porosity sufficiently high to allow skin beneath the tape to fully dry, that the material forming the tape includes polyvinyl chloride, that the thickness of the tape is less than half a millimeter, that the tape is provided on a roll with ends of the tape provided by cutting the roll, and that a tensile strength of the tape is at least about 10 newtons/centimeters.

At least one method of practicing the above invention includes a method for closing a wound in skin, including the steps of bringing margins of the wound together and into contact; and placing tape over the wound with portions of the tape in contact with skin on either side of the wound, and wherein said bringing step includes the tape having a thin flexible layer of material extending between lateral edges spaced apart by a width of the tape, and extending between opposite ends spaced apart by a length of the tape, and with the material having a lower surface parallel with and spaced from an upper surface by a thickness of the tape, wherein a non-polymerizing adhesive is located upon said lower surface, said adhesive being biocompatible with placement adjacent to human skin, said adhesive able to hold to human skin strong enough to avoid un-bonding from human skin when forces associated with wound margin spreading are encountered, and the material having a porosity greater than an average porosity of human skin, such that fluids such as blood are drawn out of the skin and into the material forming the tape.

Options for such a method as that disclosed above include the further step of allowing blood to clot within the material forming the tape, the further step of the skin being skin of a patient in excess of 80 years of age, with thin skin, the skin being skin of a patient taking a blood thinner medication, and/or wherein a coagulant agent is provided upon the material forming the tape.

### OBJECTS OF THE INVENTION

Accordingly, a primary object of the present invention is to provide a tape which can close a wound by attaching two opposing margins of the wound and applying a closing force across the wound.

Another object of the present invention is to provide a tape which is porous so that bodily fluids from a wound beneath the tape can pass into the tape at least somewhat.

Another object of the present invention is to provide a tape which is flexible so that the tape can conform to skin contours.

Another object of the present invention is to provide a tape which includes an adhesive on a lower surface thereof, which adhesive is a non-polymerizing contact adhesive which does not require a chemical reaction to occur in order to form a bond, but is ready to hold to skin upon contact.

Another object of the present invention is to provide a method for healing a cut in skin using healing tape.

Another object of the present invention is to provide a method for closing a wound using tape with a non-polymerizing adhesive on the lower surface thereof, and with the tape being more porous than human skin so that bodily fluids discharged from a wound tend to seep from the wound and harden outside of the wound.

Another object of the present invention is to provide a bandage and method which minimizes or eliminates scarring by holding margins of a wound together during healing.

Another object of the present invention is to provide a bandage and method for healing thin skin of elderly patients, without requiring sutures and minimizing or eliminating scarring.

Another object of the present invention is to provide wound/cut closure with a surface applied tape that allows unrestricted flow of blood into and across margins of the cut to promote healing.

Another object of the present invention is to provide a method and apparatus for wound/cut healing which is more rapid than suturing, and typically just 4-7 days instead of as much as 4-6 weeks or longer.

Another object of the present invention is to close wounds while avoiding the suture hole “bullet hole” type scarring.

Other further objects of the present invention will become apparent from a careful reading of the included drawing figures, the claims and detailed description of the invention.

### BRIEF DESCRIPTION OF THE DRAWINGS

FIG. 1 is a top plan view of a typical untreated cut or incision within skin, and also a partial section view taken along line A-A of the plan view of this figure.

FIG. 2 is a top plan view of a cut or incision which has been sutured closed, and including a partial section view taking along line B-B of this figure and also a partial section view taken along line C-C of this figure, section B-B located at a suture location and section C-C located at a midpoint between two sutures.

FIG. 3 is a perspective view of the healing tape of this invention being applied perpendicularly to a cut or incision within skin, the cut or incision indicated by a dashed line.

FIG. 4 is a perspective view of the healing tape of this invention being applied parallel to a cut or incision within skin, the cut or incision indicated by a dashed line.

FIG. 5 is a top plan view of a wound and showing spreading forces at margins of the wound and bodily fluid flow from the wound.

FIG. 6 is a top plan view of that which is shown in FIG. 5, but with margins of the wound closed by the healing tape of this invention, by applying a closing force.

FIG. 7 is a perspective view of a pre-cut section of tape for use according to an embodiment of this invention.

FIG. 8 is a side elevation view of a wound in skin that is closed by tape of this invention, closing the wound at the surface.

### DESCRIPTION OF THE PREFERRED EMBODIMENT

The healing tape is comprised of two components to heal cuts and incisions as well as other wounds and sores. In FIG. 1, plan view and Section AA, item 101 shows the two edges of a typical cut at the surface of skin before treatment; item 102 shows the center and deepest extent of the cut, and item 103 shows the two opposing sides of the cut.

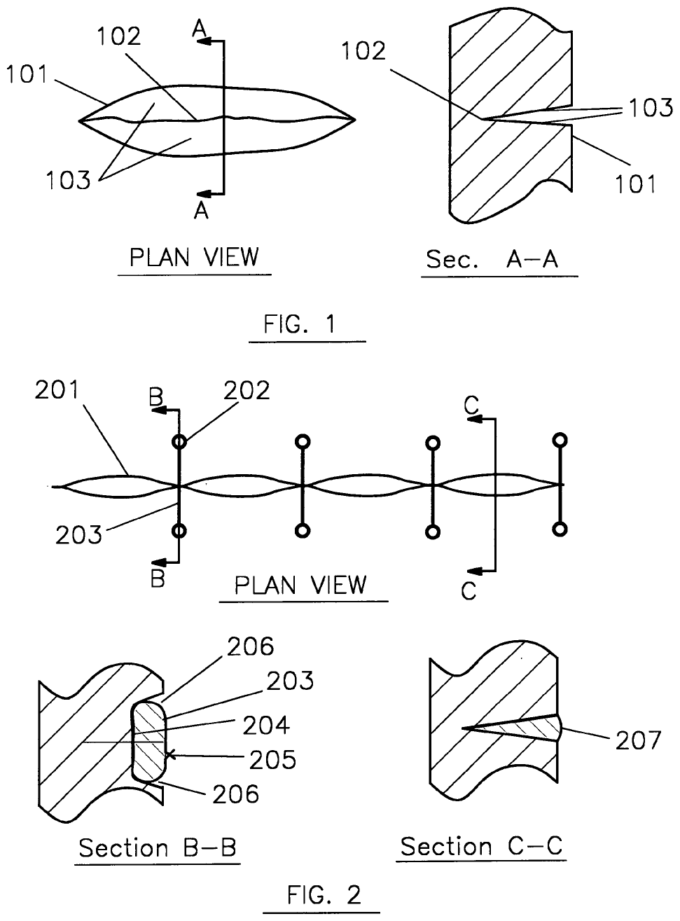

For comparison FIG. 2 shows prior and existing art, not the “healing tape” invention.

Present treatment of cuts and incisions with suturing is described using FIG. 2, plan view and sections B-B and C-C.

In the plan view, item 201 is the outside edge of a typical wound with typical sutures item 202 in place along the two edges of the cut and some distance from the edge of the cut. Section B-B shows the suture thread as item 203 on the surface and 204, the same suture within or under the surface of the skin. Item 205 shows the required surgeons knot. Each suture is a separate unit and between any two sutures the skin, which is “rubbery,” is free to create an opening for blood escape and formation of scab materials, item 207. Item 206 indicates the two openings in the surface of the skin for each suture created by the needle and pulled “open” by tension in the suture thread. Scabs and scar tissue forms in each place the needle enters or exits, 206. Section C-C, item 207 shows the scab material which forms between each pair of sutures.

Item 301, 401 is the surface of surrounding skin, 302 is the tape backing, 303 is the adhesive layer or adhesive surface. The healing tape composite, including tape backing, item 302, and adhesive 303, are applied directly to and over the cut while the two sides are pressed together.

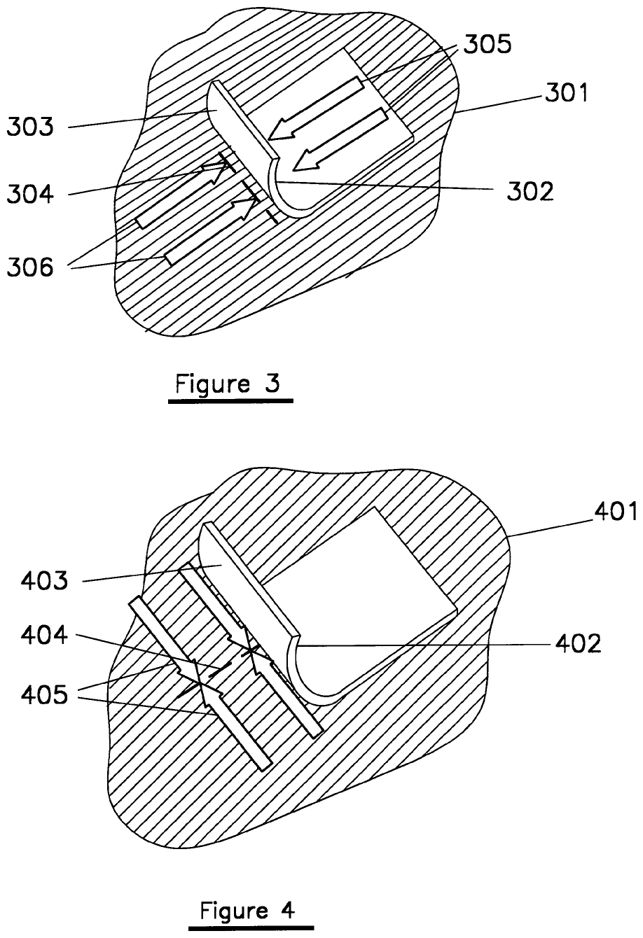

FIG. 3 and FIG. 4 show the components and typical application of the healing tape. FIG. 3 shows a cut item 304 shorter than the width of the tape, while FIG. 4 shows a longer cut with the healing tape applied lengthwise along the cut. The healing tape is comprised of a water vapor permeable tape backing (shown as items 302 and 402) and an adhesive layer, item 303 and 403. The backing defines a first surface opposite a second surface, and also generally refers to the material which makes up the tape between the surfaces. The backing being water vapor permeable and somewhat water resistant has sufficient tensile strength to resist the relative motion or shearing of the skin, is bonded to the skin on opposite sides of a cut line during healing. The second surface, the adhesive layer (items 303 and 403), with the backing are sufficiently flexible to adapt to the curved surface of the skin. Typically the tape is at least as flexible as human skin and has an elasticity that is less than that of human skin. In one embodiment, tensile strength of the tape is about 3,000 grams per inch of tape width. The healing tape holds the two sides of the cut 304 and 404 in compressive contact while capillaries can provide blood flow to all areas and surfaces of the cut, and carry away lymph fluid and blood that is depleted of oxygen and nutrients.

Application of the tape is performed in three steps. For a short cut:

Step one, a length of healing tape of up to approximately 6 centimeters long (shorter for the smallest cuts), is placed on the skin on one side of the cut as shown in FIG. 3.

Step two requires applying pressure, arrows 305 on the cut using the adhered tape on one side and at the same time, pressure a short distance from the cut as shown by arrows 306 to force the two sides of the cut into intimate contact along its full length. Light finger pressure is adequate.

Step three requires the free end of the tape to be brought into contact with the skin while the two sides of the cut remain in intimate contact.

A longer cut, item 404, is addressed in similar fashion, the difference being the application of force and the need for the tape to be approximately 6 cm longer than the cut.

Step one—the tape is applied to the skin beyond one end of the cut.

Step two—force is applied to both sides of the cut, arrows items 405 before the tape is brought into contact with the skin where the two sides of the cut are in intimate contact. If the cut is quite long, the force and application of the tape is done in a series of identical steps.

Step three—the free end of the tape is brought into contact with the skin; a distance of about two centimeters.

The adhesive layer has sufficient bonding strength to hold opposite sides of the cut in intimate contact and to remain in place during the time required for healing. Small amounts of blood may initially escape the cut and accumulate mostly within the healing tape. Initial bonding time for the two sides of the cut has been found to be approximately an hour or slightly more. Bond strength increases with time. Leaving the healing tape in place for a week to 10 days is recommended.

During this time the tape and the cut do not require any attention. Washing the area, including the tape is permissible. Heavy force or pressure on the tape are to be avoided for at least several days.

The healing tape is also useful for promoting the healing of scrapes and sores, especially as can be had for elderly people with fragile skin. The healing tape performs the function of sutures to close a wound but without the scaring and infection probability of typical sutures or stitches as they are called.

Another type of skin wound is a torn flap. Skin, especially for elders, can be snagged and torn, producing a triangular “flap” with a 60 degree to 90 degree apex. Edges of the flap can be pulled back to be in contact with normal skin and the healing tape applied for scarless healing.

Wounds which remove an area of skin can also be protected with an application of the healing tape. The missing skin is replaced with natural skin, without using additional scaffolding type skin. Healing time for a torn flap is longer than for cuts depending on the dimensions of the missing skin.

Results of using healing tape are rapid reduction in pain; often to no pain as the tape is applied. Rapid reduction in bleeding; often no bleeding or excess bleeding. Elimination of scab formation if the two sides of the cut are in direct contact. Scabs and scar tissue cells are not formed. Instead pink skin type cells are formed which become normal color matching adjacent skin within weeks. Reduction in infections; usually to no infections. Elimination of the need to redress wounds. Since no sutures are used, elimination of the need to remove sutures. Elimination, or near elimination of scars; depending on care to close the wound.

The method of healing with healing tape can also be used on a wound that is a void in the surface of the skin; the void being preferably less than one half inch in diameter and one-eighth inch deep. In this type of wound, there are no opposite surfaces to push into contact. The steps of healing include: cleaning the void and the skin around the void if possible, applying a strip of healing tape over the cut, leaving the tape in place for over much longer than one week, the length of time determined by the width and depth of the void. Healing a void is much slower than healing a cut or tear or even a sore. Healing tape has been used to heal voids, new skin material forms under the tape without scaffolding material being used. In the observed use, new skin filled the void without leaving a scar.

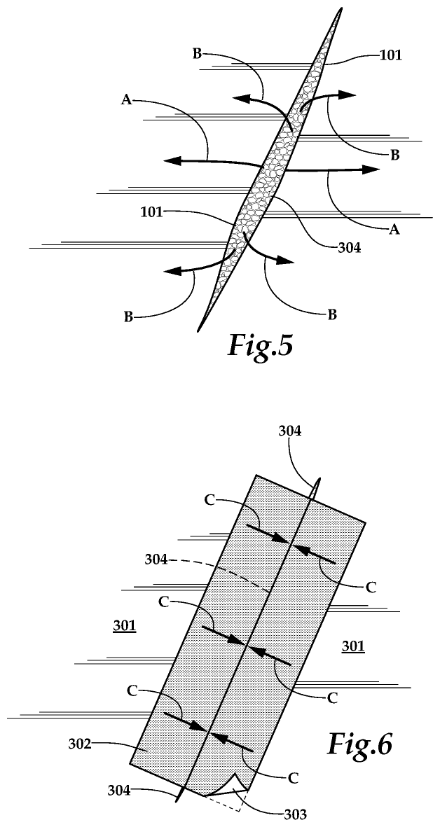

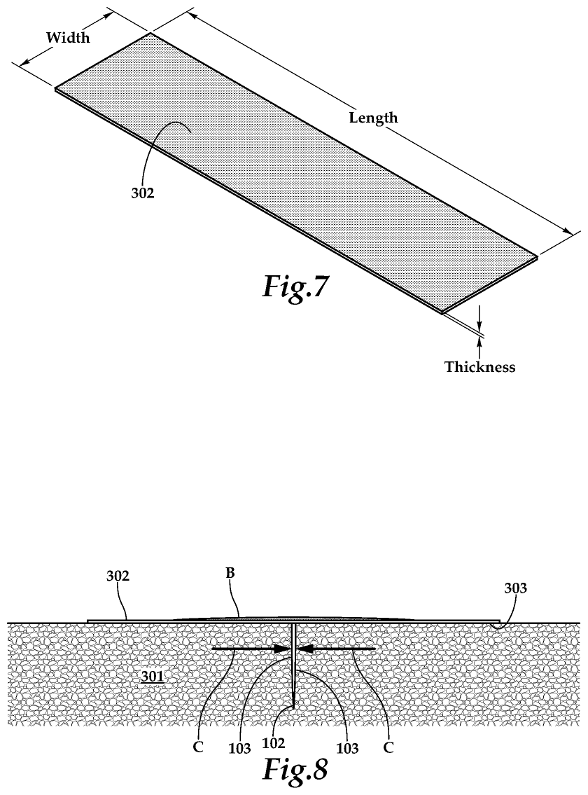

FIGS. 5-8 show a wound before treatment and with the wound exhibiting a spreading force along arrow A (FIG. 5) and with bodily fluids B, such as blood discharging from the wound. In FIG. 6 the tape has been applied and a closing force, along arrow C, is holding the wound closed. FIG. 7 shows how width, length and thickness of the tape relate to each other. In one embodiment, the thickness is less than half a millimeter and the width is one-half inch, or three-quarter inch or one inch. The length can vary and is either cut to a desired length off of a spool/roll or provided in pre-cut length. FIG. 8 shows how the wound appears in elevation and how the tape closes the wound and allows bodily fluids to pass out of the wound in into or above the tape.

An adhesive 303 is shown in FIGS. 6 and 8 (as well as in FIG. 3). This adhesive is a contact adhesive that is non-polymerizing (does not need a polymerizing reaction to take place to have adhesive properties). Examples of this adhesive include methacrylate, polychloroprene, silicone, with or without compatible modifying resins. Other adhesive options include phenolics, terpene phenolics or phenolic-modified rosin esters; or silicone gel, acrylic or hydrogel adhesives.

This disclosure is provided to reveal a preferred embodiment of the invention and a best mode for practicing the invention. Having thus described the invention in this way, it should be apparent that various different modifications can be made to the preferred embodiment without departing from the scope and spirit of this disclosure. When embodiments are referred to as “exemplary” or “preferred” this term is meant to indicate one example of the invention, and does not exclude other possible embodiments. When structures are identified as a means to perform a function, the identification is intended to include all structures which can perform the function specified.

What is claimed is:

1. A method for healing a cut in skin using healing tape comprising the steps of:
closing the cut so that the opposite sides of the cut are brought back into proximity and contact;
covering the cut with healing tape so that with the opposite sides of the cut are maintained in contact, the healing tape including a first surface comprised of a water vapor permeable tape backing, and a second surface comprised of a skin compatible adhesive;
pressing the adhesive on the second surface of the tape to the skin on opposite sides of the cut;
the cut becoming immobilized by the attachment of the healing tape to the skin as a result of the mechanical strength of the healing tape in tension and in shear; and
wherein the adhesive is a non-polymerizing adhesive.

2. The method of claim 1 where the opposite sides of the cut are caused to close by applying the healing tape to a first side of a cut, then applying a tension to the tape so that the flesh is caused to close the cut according to said closing step, and then pressing the tape against the skin on the opposite side of the cut so that the tape is adhesively bonded to both sides of the cut.

3. The method of claim 1 where the water vapor permeability of the tape is larger than a water vapor permeability value of skin beneath the tape, such that the skin avoids becoming water saturated by the tape.

4. The method of claim 1 where the cut is cleaned before the tape is applied.

5. The method of claim 1 where an anti-bacterial agent is applied to the healing tape prior to its application to the skin.

6. The method of claim 1 where the tape is cut from a continuous spool of tape.

7. The method of claim 1 where the cut results from surgery and the healing tape is applied to secure and close the wound post surgery.

8. The method of claim 1 where the opposite sides of the cut are pushed together with fingers prior to application of the healing tape.

9. The method of claim 1 wherein the adhesive is a contact adhesive which releasably bonds to human skin.

10. The method of claim 9 wherein said adhesive includes a methacrylate.

11. The method of claim 1 wherein the tape has a thickness of less than about half a millimeter.

12. The method of claim 1 wherein the tape is formed primarily of polyvinyl chloride.

13. The method of claim 1 where the wound is a tear.

14. The method of claim 1 wherein the healing tape is provided in precut lengths.

15. A method for healing a cut in skin, including the steps of:
wiping debris away from the cut;
drying skin on either side of the cut;
comparing a length of the cut to a width of healing tape, the healing tape comprised of a flexible tape backing on a first surface and a tape adhesive on a second surface opposite the first surface, the healing tape having an elongate form with a length greater than the width;
attaching the healing tape to skin adjacent to the cut with the healing tape oriented with a long axis perpendicular to the cut if the cut is determined by said comparing step to be shorter than the width of the healing tape or the healing tape oriented with a long axis parallel to the cut if the cut is determined by said comparing step to be longer than the width of the healing tape;
said attaching step including attaching a first end of the healing tape to the skin through the adhesive on the second surface, closing the cut, laying the healing tape over the cut, and attaching a second end of the healing tape to the skin opposite the first end of the healing tape, through the adhesive on the second surface;
wherein the healing tape of said comparing step exhibits flexibility at least as flexible as skin flexibility;
wherein the healing tape of said comparing step exhibits elasticity less than elasticity of the skin;
wherein the flexible backing of the healing tape of said comparing step is water vapor permeable to a greater degree than skin, such that water vapor exiting skin is not trapped against a surface of the skin beneath the backing; and
wherein the healing tape adhesive is a non-polymerizing adhesive.

16. A method for closing a wound in skin, including the steps of:
bringing margins of the wound together and into contact; and placing tape over the wound with portions of the tape in contact with skin on either side of the wound; and
wherein said bringing step includes the tape having a thin flexible layer of material extending between lateral edges spaced apart by a width of the tape, and extending between opposite ends spaced apart by a length of the tape, and with the material having a lower surface parallel with and spaced from an upper surface by a thickness of the tape, wherein a non-polymerizing adhesive is located upon said lower surface, said adhesive being biocompatible with placement adjacent to human skin, said adhesive able to hold to human skin strong enough to avoid un-bonding from human skin when forces associated with wound margin spreading are encountered; and the material having a porosity greater than an average porosity of human skin, such that fluids such as blood are drawn out of the skin and into the material forming the tape.

17. The method of claim 16 including the further step of allowing blood to clot within the material forming the tape.

18. The method of claim 16 including the further step of the skin being skin of a patient in excess of 80 years of age, with thin skin.

19. The method of claim 16 wherein the skin being skin of a patient taking a blood thinner medication.

20. The method of claim 16 wherein a coagulant agent is provided upon or within the material forming the tape.
